(19) United States
(12) Patent Application Publication
(10) Pub. No.: US 2010/0312136 A1
(43) Pub. Date: Dec. 9, 2010

(54) SYSTEMS AND METHODS FOR THERMOGRAPHIC-GUIDED MEDICAL TREATMENT

(75) Inventor: Christopher Cozzie, Cardiff, CA (US)

Correspondence Address: PROCOPIO, CORY, HARGREAVES & SAVITCH LLP, 525 B STREET, SUITE 2200, SAN DIEGO, CA, 92101, US

(73) Assignee: ADVANCED FUNCTIONAL SYSTEMS, LLC, Cardiff, CA (US)

(21) Appl. No.: 12/648,213

(22) Filed: Dec. 28, 2009

Related U.S. Application Data
(63) Continuation-in-part of application No. 12/144,571, filed on Jun. 23, 2008, now abandoned.
(60) Provisional application No. 60/945,877, filed on Jun. 22, 2007.

(51) Int. Cl. A61B 5/01

(52) U.S. Cl. 600/549

(57) ABSTRACT

A system for detecting the risk of breast cancer comprises a thermographic imaging device configured to acquire thermographic images of a female torso; an analysis authority communicatively coupled with the thermographic imaging device, the analysis authority including an input port configured to receive thermographic images from the thermographic imaging device, a database configured to store the thermographic images, a qualitative analysis module configured to automatically perform a qualitative analysis of the thermographic images and generate a qualitative score based on the qualitative analysis, a quantitative analysis module configured to automatically perform a quantitative analysis of the thermographic images and generate a quantitative score based on the quantitative analysis, and a scoring module configured to correlate the qualitative and quantitative scores with a score indicating a risk of breast cancer.

### RELATED APPLICATIONS INFORMATION

This application claims the priority as a Continuation-In-Part under 35 U.S.C. 120 to U.S. patent application Ser. No. 12/144,571, filed Jun. 23, 2008, and entitled “Methods of Thermographic-Guided Medical Treatment,” which in turn claims the benefit under 35 U.S.C. 119(e) of U.S. Provisional Application No. 60/945,877 entitled “Methods of Thermograph-Guided Medical Treatment” and filed Jun. 22, 2007, both of which are incorporated herein by reference as part of the specification of this application.

### BACKGROUND

1. Technical Field

The embodiments described below relate to the use of thermography to diagnose disease and monitor treatment, and in particular the use of thermography to diagnose breast cancer and to monitor the treatment thereof.

2. Related Art

Infrared Thermography, thermal imaging, thermographic imaging, or thermal video, is a type of infrared imaging science. With the improvement in thermographic cameras, it is possible to devise systems and methods to allow early detection of various diseases and conditions. For example, when combined with the appropriate evaluation protocol and techniques, thermography can be used for early detection of breast cancer, as explained below; skin cancer; vascular disease; certain neurological disorders.

Thermographic cameras detect radiation in the infrared range of the electromagnetic spectrum, roughly 900-14,000 nanometers or 0.9-14 μm, and produce images of that radiation, called thermograms. The word Infrared is generally used to describe a portion of the electromagnetic spectrum with wavelengths between one micrometer and one millimeter, that is perceived as heat. These wavelengths are longer than those of visible light but shorter than those of microwaves or radiowaves. The amount of radiation emitted by an object increases with temperature, therefore thermography allows one to see variations in temperature. When viewed by thermographic camera, warm objects stand out well against cooler backgrounds.

One area in which thermography is beginning to gain acceptance as a diagnostic tool is in the area of thermographic, or infrared mammography. The term Infrared Mammography is used to describe the process of making diagnostic-quality images of the breasts' radiant infrared energy for the detection of cancer.

Thermology is the medical science that derives diagnostic indications from diagnostic-quality infrared images of the human body by the use of highly-resolute and sensitive infrared (thermographic) cameras. Infrared Mammography utilizes the principles of thermology as a diagnostic technique in the early detection of breast cancer in a clinical setting or as a monitor of its treatment. Infrared Mammography is completely non-contact and imparts no form of radiation energy onto or into the body.

The development of new blood vessels into a solid cancerous neoplasm must occur when it has grown too large for the metabolic needs of the cells at the tumor's center to be met by diffusion from existing blood vessels. Practically, the process of developing new blood vessels, called neo-angiogenesis, is necessary for a cancerous tumor to grow larger than about 150 micrometers (0.15 mm) in diameter and must be extensively developed by the time a cancerous tumor has grown to two mm (approx. ⅛″) in diameter. The luminal ultra-structure of cancerous neo-angiogenetic blood vessels is aberrant and there is an absence of any integration of these vessels with the autonomic nervous system. Further, Nitric Oxide (NO) is a small uncharged molecule that easily diffuses into adjacent tissues with a very potent regional vaso-dilatory effect. NO is produced in abundance by biochemical pathways of the ferritin formation associated with cancer. These two cancer-related factors both lead to the unregulated hyperemia of core-body temperature blood into the proximity of breast cancer and are responsible for the characteristic increases in skin temperature overlying breast cancer. Sound diagnostic methodology necessarily involves some form of physiologic-based dynamic differentiation of this unregulated blood flow in order to indicate cancer. If appropriate diagnostic protocols are developed, then Infrared Mammography can provide a high diagnostic sensitivity and good specificity.

Using similar principles, thermography can be used to diagnose other diseases and conditions, such as those described above; however, developing the appropriate diagnostic protocols and techniques can be easier said than done.

### SUMMARY

A system for the identification of a risk of a certain type of breast cancer based on thermographic images, as well as identification of a treatment process, and monitoring the effectiveness of the treatment process is disclosed herein.

A system for detecting the risk of breast cancer comprises a thermographic imaging device configured to acquire thermographic images of a female torso; an analysis authority communicatively coupled with the thermographic imaging device, the analysis authority including an input port configured to receive thermographic images from the thermographic imaging device, a database configured to store the thermographic images, a qualitative analysis module configured to automatically perform a qualitative analysis of the thermographic images and generate a qualitative score based on the qualitative analysis, a quantitative analysis module configured to automatically perform a quantitative analysis of the thermographic images and generate a quantitative score based on the quantitative analysis, and a scoring module configured to correlate the qualitative and quantitative scores with a score indicating a risk of breast cancer.

According to another aspect, a method for detecting the risk of breast cancer, in a system comprising a thermographic imaging device configured to acquire thermographic images of a female torso and an analysis authority communicatively coupled with the thermographic imaging device comprises receiving thermographic images from the thermographic imaging device; storing the thermographic images in a database; automatically performing a qualitative analysis of the thermographic images; generating a qualitative score based on the qualitative analysis; automatically performing a quantitative analysis of the thermographic images; generating a quantitative score based on the quantitative analysis; and correlating the qualitative and quantitative scores with a score indicating a risk of breast cancer.

These and other features, aspects, and embodiments are described below in the section entitled “Detailed Description.”

### BRIEF DESCRIPTION OF THE DRAWINGS

Features, aspects, and embodiments are described in conjunction with the attached drawings, in which:

FIG. 1 is a diagram illustrating an example system for acquiring and analyzing thermographic images in accordance with one embodiment;

FIG. 2 is a flow chart illustrating an example process for acquiring thermographic images and performing analysis thereon using the system of FIG. 1 in accordance with one embodiment;

FIG. 3 is a diagram illustrating an example thermographic image acquired using the system of FIG. 1;

FIG. 4 is a flow chart illustrating an example process for acquiring thermographic images and performing analysis thereon to detect a risk of breast cancer using the system of FIG. 1 in accordance with one embodiment;

FIG. 5 is a flow chart illustrating an example process for identifying a treatment and monitoring the effectiveness of the treatment using the system of FIG. 1 in accordance with on embodiment; and

FIG. 6 is a flow chart illustrating an example process for identifying a treatment and monitoring the effectiveness of the treatment using the system of FIG. 1 in accordance with another embodiment.

### DETAILED DESCRIPTION

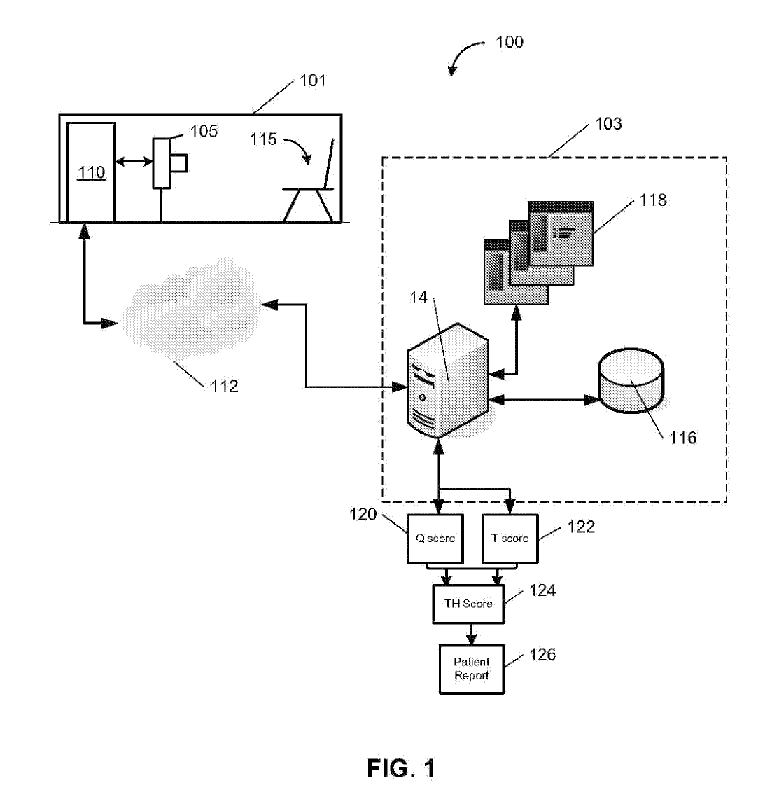

FIG. 1 depicts a schematic of an example system 100 for thermographic imaging, the detection of an evolving disease process, identification of a treatment regime, and monitoring of treatment results. System 100 includes an enclosure 101 for thermographic imaging and an analysis authority 103. The enclosure 101 is used for thermographic imaging, and as such the parameters of the enclosure 101 should adhere to particular standards so that the thermographic images are accurate and dependably reproducible. The parameters of the enclosure 101 can include enclosure dimensions, enclosure temperature, enclosure pressure, and the like. Although one particular example of an enclosure 101 is depicted in FIG. 1 and one particular set of enclosure parameters are specified, other enclosures having other dimensions are also possible depending upon factors including available space, image properties, patient conditions, and the like.

In some implementations, the enclosure 101 is large enough for one technician and one patient. In such implementations, the floor plan of the enclosure 101 can be 8′×8′. In other implementations, the enclosure 101 can be larger to accommodate more people, and the floor plan in such implementations can be, e.g., 10′×12′, or larger. The enclosure 101 can include a thermographic imaging device 105, e.g., an infrared camera, to capture thermographic images of a patient. The thermographic imaging device 105 can be operatively coupled to a thermographic image processing device 110 to which thermographic images captured by the thermographic imaging device 105 are transferred. In an alternative embodiment, device 105 can simply transfer images to authority 103.

The enclosure 101 includes a patient seating area 115 that can be a pre-determined distance from the thermographic imaging device 105. The floor of the enclosure 101 can be carpeted entirely or at least in the patient seating area 115. A grid or other marker can be used to ensure that the distance between the thermographic imaging device 1050 and the patient seating area 115 remains constant to maintain consistency of each thermographic image captured. For example, the thermographic imaging device 105 can be 3 feet from the patient seating area 115. In some implementations, the distance between the thermographic imaging device 105 and the patient seating area 115 can be sufficient to fill a screen of the thermographic imaging device 105 with an image of the patient. In other implementations, the distance can be determined such that an area being imaged is visible in the screen of the thermographic imaging device 105.

In some implementations, the position of the patient can be altered to capture thermographic images of an area of interest from different angles. For example, depending on the types of images and areas of interest, the patient may be lying down or standing up; the patient's, front, back, or side may be facing the camera; the camera, or the patient relative thereto, may even be positioned so as to obtain images of the top of the individual's head or the bottom of their feet. For example, in a breast imaging application, a first image, called an anterior image, can be captured with the area of interest facing the thermal imaging device 105. A second image, called a left oblique image, can be captured where a thermographic image of the left-most region of the area of interest is captured. A third image, called a right oblique image, can be captured where a thermographic image of the right-most region of the area of interest is captured. The thermographic analysis can be performed using all the thermographic images. Although three examples of thermographic images are described, thermographic images can be captured by positioning the area of interest at any angle to the thermal imaging device 105.

In such an application, the patient seating area 115 can include a seating device, e.g., chair, stool, and the like, that can be swiveled to enable capturing anterior, left oblique, and right oblique images of the patient. Additional markers can be included in the grid on the floor of the enclosure 101 that enable identifying the angle by which the chair, table, etc., can be swiveled to achieve the appropriate angle. For example, an outline of two feet can be drawn to the left and to the right of the chair. The patient can be instructed to position both feet in the markers to the left and the right of the chair to capture a right oblique image and a left oblique image, respectively.

The enclosure 101 can include thermostats or thermometers located on all the walls of the examination room approximately at a height of 4′-6′ from the floor. In some implementations, the enclosure 101 can include four walls, and consequently, four thermostats or thermometers. In some implementations, more than one thermostat or thermometer can be used on the same wall. In implementations where the enclosure 101 includes more than four walls, additional thermostats or thermometers can be used.

The enclosure 101 can be maintained within an optimal temperature range for the thermographic imaging. For example, the enclosure 101 temperature can be 20° C. (68° F.), and the enclosure 101 can be maintained in a temperature ranging from 18.0° C. to 22.0° C. The thermostat or thermometer should be programmable to control the temperature within the enclosure 101 so that ambient temperature in the examination room does not change more than 1° C. during the examination.

Additional methods to maintain patient temperature during examinations can include ensuring that air condition ducts do not blow on the patients, and ensuring that the patient is not near exterior windows. This can be achieved, e.g., by using air deflectors on the air ducts or foam core at exterior windows. The height of the seating device and the thermographic imaging device 105 can be adjustable for the patient's comfort such that the patient's feet are flat on the floor while sitting in the seating device. The seating device can include a cover made of cloth or leather or the like. The enclosure 101 can also include a room divider such as a curtain or panel. In addition to acting as added privacy for the patient, this divider can be used between the patient and the technician/equipment to control auxiliary release of heat that can affect the accuracy of the images captured.

Prior to the patient entering the enclosure 101, the temperature of the enclosure 101 should be stabilized and maintained constant at approximately 20±2° C. Neutral temperature for a resting patient is approximately 30° C. An enclosure 101 temperature range of 18° C. to 22° C. can create a cooling effect without causing the patient to shiver. Shivering can cause blood flow to be directed back to the skin as a protective response to the cold environment and can interfere with thermographic imaging resulting in false positive findings. The temperature in the room should create a negative heat load causing more heat to leave the patient's body than the heat being made by the patient's body. This condition is considered to be optimum for obtaining the best thermographic images.

The temperature of the enclosure 101 can be checked at different time instants during thermographic imaging to ensure that the temperature variation during the imaging is within a threshold, e.g., ±1° C. or ±0.5° C. In some implementations, the temperature can be checked when certain events occur. For example, the temperature can be checked prior to patient entry, prior to thermographic image captures, and prior to performing each step of the thermographic imaging process. In other implementations, the temperature can be checked at regular time intervals. In other implementations, temperature checking can be a combination of both.

The enclosure 101 can include a thermographic image processor 110 that is operatively coupled to the thermographic imaging device 105. Processor 110 can be configured to receive images captured by device 105 and to forward the images to authority 103. In certain embodiments, processor 110 can be configured to store the images. For example, processor 110 can be a computer or server interfaced with device 105. In other embodiments, processor 110 can be integrated with device 105. In other embodiments, processor 110 can be omitted or included in authority 103.

In some implementations, the thermographic image processor 110 can be within the enclosure 101 and the heat generated by the processor 110 can be considered while maintaining temperature of the enclosure 101. In other implementations, the thermographic image processor 110 can be positioned outside the enclosure 101 and operatively coupled to the thermographic imaging device 105 through wired or wireless means. For example, the thermographic imaging device 105 and the thermographic image processor 110 can be operatively coupled via one or more networks, where the imaging device 105 can transmit the captured images to the processor 110 for further processing, e.g., printing.

Processor 110, or device 105 depending on the embodiment can be interface with message authority 103 through a network or networks 112. For example, processor 110 or device 105 can be interfaced with authority 103 through the Internet. In such implementations, images can be captured by device 105 and transferred to processor 110, where they can be stored. Processor 110 can then be used to access a website associated authority 103 and to upload the stored images to the website. Other information can be transferred as well, such as patient information, physical examination information, and photographs.

Network 112 can comprise one or more wired or wireless Local Area Networks (LANS), one or more wireless Wide Area Networks (WANs), one or more wired or wireless Metropolitan Area Networks, (MANs), etc.

Analysis authority 103 can comprise the hardware and software resources to perform the functions disclosed herein. As such, authority 103 can comprise the servers, routers, user interfaces, APIs, software routines and modules, inputs, outputs, storage, etc. required to perform the functions disclosed. In particular, authority 114 can comprise one or more servers configured to receive the images uploaded by processor 110 or device 105 as well as sufficient storage 116 to store the images. Authority 103 can also comprise one or more software programs, routines, modules, algorithms, etc., collectively modules 118, for processing the images and automatically performing qualitative and quantitative analysis of the images.

Modules 118 can be further configured to generate qualitative scores, Q scores 120, and quantitative scores, T scores 122, which can correspond the a thermal score, TH score 124, and to generate a report 126 that can be sent back to processor 110 or stored and accessed on line by processor 110.

Modules 118 can be hosted on server(s) 114 and accessed via remote terminals, or can be stored on the remote terminals (not shown). In either case, a technician can access modules 118 through a terminal, pull up a set of images, and cause the quantitative and qualitative analysis to be performed as explained below. It will be understood that a terminal can comprise a computer, laptop, mobile computing device, or dumb terminal depending on the embodiment. Thus, each terminal can have a set of modules loaded thereon, or can have some form of thin client that allows access to modules 118 on server 114.

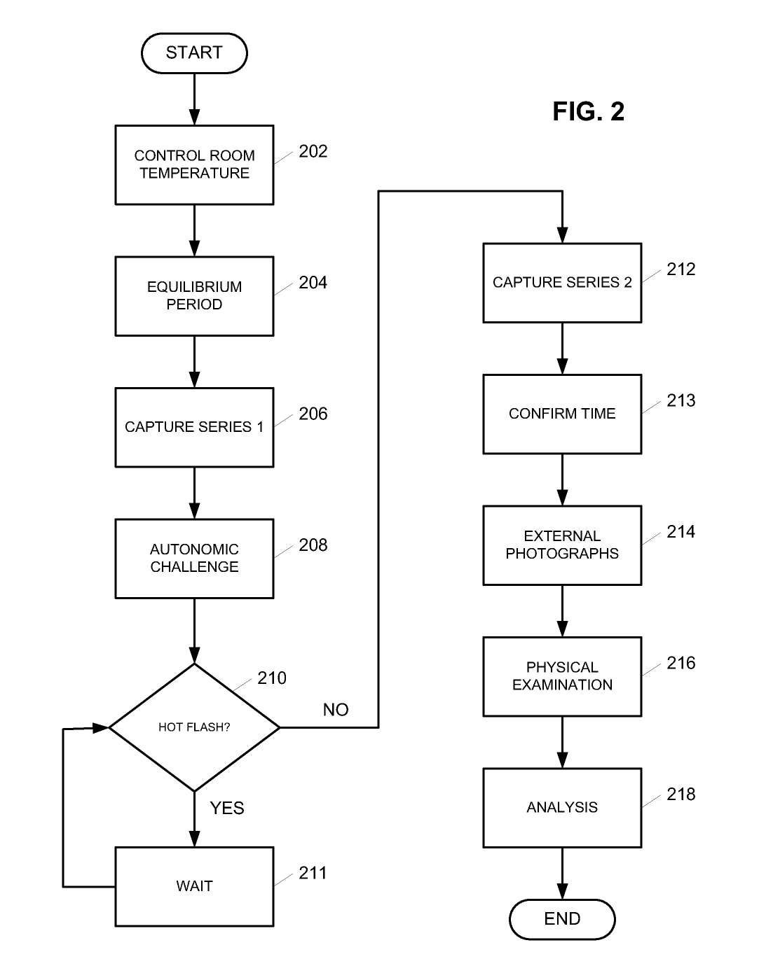

FIG. 2 is a flow chart illustrating one example process for acquiring and analyzing thermal images in accordance with one embodiment. First, as noted above, the temperature in enclosure 101 can be controlled in step 202. Once the patient enters the enclosure 101, the temperature equilibration period commences in step 204 and should last for a duration, e.g., 15 to 20 minutes. Additional steps (not shown) for temperature equilibration can include removing jewelry and clothing of the upper body, or body part being imaged, including shoes and socks/stockings, pinning up of long hair, wearing gowns that do not have a plastic lining, and the like. The physician as well as the patient should avoid contact with the skin of the patient's chest, or area being imaged. No prodding or physical exams of the chest, or area being imaged, should occur during the equilibration period.

For breast imaging, areas of the chest imaged by thermography include the region from the supraclavicular fossa to the lower breasts, mid and upper chest and both axillary regions. These areas of the patient's chest should remain uncovered during imaging and contact to these areas should be avoided not only during the equilibration period but also during the entire examination period.

Directly following the 15 to 20 minutes of equilibration, the thermographic imaging device 105 can be used to capture in step 206 a first series of thermograph images (Series 1 images) of the patient's, e.g., breast region that serve as a baseline. If the patient wore a gown during equilibration, it must be removed during image capture. In this example, the Series 1 images includes one image of anterior breasts (anterior image) where the patient faces directly at the camera, one image of left oblique breast (left oblique image) where the patient is turned 45° to the right and one image of the right oblique breast (right oblique image) where the patient is turned 45° to the left. For each image captured, the patient should position themselves with straight posture, feet flat on the floor, and their hands either on the top or the back of the head. If a patient is unable to position their hands on their head, the hands can alternatively be on the hips.

An autonomic challenge, also referred to as a provocative challenge or stress test, can be performed in step 208 following capture of Series 1 images. For example, in one embodiment, the patient soaks their hands in a basin of cold water for one minute. The temperature of the water can be a pre-determined temperature, e.g., 10° C., and should be cold relative to the room and the patient's body temperature. Although the specific temperature of the water in the basin is not critical, it should be at least cold enough to cause an autonomic response. The depth of the water in the basin should be sufficient to cover at least the backs of the hands. After soaking the hands in the cold water, the hands can be dried for a period of time. A person operating the thermographic imaging device 105 can confirm that the patient experienced no hot flashes in step 210 during this period. If a hot flash occurred, the technician can wait, e.g., 10 minutes in step 211 prior to initiating the next round of thermographic image capture of images, namely, Series 2 images in step 212. Series 2 images are captured in a manner similar to the Series 1 images.

Following capture of the Series 2 images, the technician confirms in step 213 that the time of examination was within the time-frame allotted for the examination. Examination should last no longer than a pre-determined period of time, e.g., 60 minutes, but this time can vary. For example, the patient's weight can increase the length of time allowed for examination up to 60 minutes. Obese patients have a longer time period during which the examination can take place.

External photographs of the patient can be captured at this time (step 214) to provide evidence for any surface abnormalities that could affect thermal patterns or to assist in selecting regions for analysis. The physician can also complete any physical examination of the patient during this time (step 216) as well as the remaining patient history and diagnosis. Although the example process for thermographic imaging describes two series of photographs captured using similar methods, additional series can be captured. For example, three or more series of photographs can be captured for qualitative and quantitative analysis.

As described above, the thermographic images captured can provide two series of, e.g., three images each, anterior, left oblique, and right oblique. Series 1 images taken prior to the autonomic challenge can act as a baseline, and Series 2 images taken after the autonomic challenge can provide functional data. Analysis (step 218) of the images requires observation of qualitative vascular patterns as well as quantitative measurement of temperature patterns. These are each based on a correlation with the vascular anatomy of the normal, e.g., breast. Regular thermovascular patterns normally correspond to one or several of the physiological venous plexuses. The quantitative and qualitative analysis of the thermal images can be aided by the photographs, to determine the likelihood that a certain condition or state exists.

Prior to analyzing the images (step 218), the ambient temperatures during the imaging steps 206 and 212 are compared to ensure that the temperature was maintained according to the proper examination protocol. In one example, the thermostats and thermometers on the walls of the enclosure 101 can be operatively coupled to the thermographic image processing device 110 and the temperatures measured by the thermostats and the temperatures can be used to ensure that the ambient temperature variations were within pre-defined thresholds.

In addition, temperature measurements of the image background can also be used to confirm proper ambient conditions. For example, temperatures of all points in a Series 1 image can be compared with the same points in the corresponding Series 2 image to obtain information regarding the temperature conditions during the course of the examination. If all the points get hotter from Series 1 to Series 2, it can be decided not to continue with the image analysis due to improper ambient conditions.

Comparison of Series 1 to Series 2 thermographs can also identify points of interest in the thermograph that might suggest further quantitative analysis. Patient intake forms and external photographs of the patient can also provide useful information that could affect or explain the observed thermal patterns. For example, scars, or other external features can contribute to the presence of abnormal thermal emission patterns. Prior knowledge of these external features can clarify and assist in interpretation of any unusual thermal features that appear in the images.

The Series 1 and Series 2 images are set to thermal windows that provide the appropriate resolution for the analysis to be performed. The thermal window or thermal focus indicates the resolution of the range of temperatures visible in the image. A 10° C. thermal window can provide, for example, a temperature range between 26° C. and 36° C. and can be useful for the overall qualitative assessment of the thermographs. A more narrow thermal focus such as 5° C. can be used to more clearly resolve a thermovascular pattern for performing qualitative analysis. Qualitative analysis can be performed viewing the images in grayscale to assign a Q score to an image, while the quantitative analysis of the temperature patterns can be performed viewing the images in color to assign a T score to an image.

Qualitative analysis includes categorizing the thermograph based on the appearance of thermovascular patterns and/or physical features to obtain a Q score using a black and white or gray scale image. A general overview of the images is performed to identify any feature or pattern that appear abnormal and warrant further quantitative analysis, e.g., asymmetric images. Normal breast thermographs have a relatively symmetrical and uniform temperature distribution over both breasts. Usually the breasts appear avascular or have only discrete vascular patterns near the upper and outer regions of both breasts. If a thermal pattern or temperature is seen on one breast, but not on the mirror area of the contralateral breast, further investigation can be performed with specific quantitative temperature measurements. In some implementations, the qualitative analysis can be performed prior to the quantitative analysis in order to identify potentially abnormal features of the thermographic image. Therefore, in addition to providing a numerical score that contributes to the patient's overall risk assessment, the qualitative findings can act as a guide for what quantitative analysis and temperature measurements should be additionally performed.

For example, in certain breast implementations there are 12 thermovascular patterns and/or physical features that can be characterized Q-1 through Q-12 and scored. Q scores can range from 0 for a normal thermograph to a maximum of 60 for the highest single score for a particular feature. A normal breast thermograph shows a symmetrical and uniform temperature distribution over both breasts. Normal breast thermographs are characterized as Q-1 and carry a score of 0. Certain qualitative thermovascular patterns can be considered to be primary risk factors for malignancy. Examples of the four qualitative features that are primary risk factors for malignancy include distorted thermovascular patterns (Q score=40), anarchic thermovascular pattern (Q score=60), distorted thermovascular outline (Q score=45), asymmetrical hypervascularization (Q score=40). If any of these features are identified, the patient should undergo follow-up thermographic examination and any appropriate additional diagnostic testing such as mammography, ultrasound, x-ray, MRI, sestamibi, ductal lavage, PET scan, blood tests, biopsy and the like.

One or more regular thermovascular patterns over a limited area of one or both breasts in the thermograph are characterized as Q-2 and carry a score of 20. The vascular patterns can be serpentine, arborescent or curvilinear. The patterns often extend downward from the upper medial region of the breast toward the nipple/areolar region. The thermal variable tends to approximate normal venous plexus distribution.

Multiple curvilinear thermovascular patterns forming a relatively symmetrical network in the thermograph can be characterized as Q-3 and can carry a score of 10. The network usually appear bilaterally at the upper breasts, and can be suggestive of early pregnancy or hormone imbalance such as estrogen/progesterone, and more rarely can appear over the entire breast, which is suggestive of advanced pregnancy or nursing mother.

Irregular thermovascular patterns positioned over a specific region of the breast in the thermograph can be characterized as Q-4 and can carry a score of 25. These irregular patterns do not necessarily correspond to the physiological venous plexus.

Distorted thermovascular patterns over a limited area of one or both of the breasts in the thermograph can be characterized as Q-5 and can carry a score of 40. The patterns are most often seen at the upper medial and upper outer quadrants and can occasionally have a hazy appearance. This type of pattern can be suggestive of fast growing tumors, mostly carcinomas or possibly evolutive fibroadenomas and cystosarcoma phyllodes. This feature is considered a primary risk factor for malignancy.

Anarchic thermovascular patterns in the thermograph can be characterized as Q-6 and can carry the highest single score for a particular feature, which is 60. The patterns are distorted and normally appear over a limited area of one breast. There are clear discontinuities in the course and caliber of the patterns. This feature is considered to be a primary risk factor for malignancy.

The presence of one or more well-defined distinct regions of hyperthermia in the thermograph can be characterized as Q-7 and can carry a score of 20. The regions can occur anywhere on the breast and have variable size and shape. Following an autonomic test, the regions typically cool. If the region warms, the score is higher than 20.

The presence of one or more well-defined distinct areas of hypothermia in the thermograph can be characterized as Q-8 and can carry a score of 15. The regions can occur anywhere on the breast and have variable size and shape. The region can also extend over the entire breast and have a mottled appearance. This pattern is often associated with cystic and fibrocystic breasts as well as benign fibroadenoma cysts. Multiple regions of hypothermia on the same breast can still only be scored as a 15.

Asymmetrical thermovascular patterns compared to the contralateral breast in the thermograph can be characterized as Q-9 and can carry a score of 15. Typically the thermovascular pattern involves only one breast. Exemplary patterns include a bifurcated vascular peduncle, inverted V, transverse pattern, and thermal asymmetry.

Distorted thermovascular outline in the thermograph can be characterized as Q-10 and can carry a score of 45. This can be a bulge in the contour of the breast that is backed by heat. In addition, the contour can be concave or a flat area called an “edge sign.” These distorted contours are typically observed with an actual anatomical distortion that can be seen on the external photograph. This feature is considered to be a primary risk factor for malignancy.

Curvilinear thermovascular patterns in the mid-chest and/or upper breasts in the thermograph can be characterized as Q-11 and can carry a score of 25. The patterns typically form a regular network and are most often symmetrical. These patterns can indicate pregnancy, nursing or an estrogen/progesterone imbalance such as low progesterone or estrogen dominance.

Asymmetrical hypervascularization of one breast compared to the contralateral breast in the thermograph can be characterized as Q-12 and can carry a score of 40, for example, an asymmetry over 25% of an area of one breast compared to the mirror region on the contralateral breast. This feature is considered to be a primary risk factor for malignancy.

Having established a Q score for the thermographic images based on a qualitative analysis, quantitative analysis can be performed by viewing the images in color to associate a T score with each image. The thermal window selected is typically 5° C. Each image captured undergoes a variety of temperature measurements that contribute to the T score. One or more points and/or one or more regions of the thermographic image that are selected for measuring temperature and calculating temperature differences is crucial to obtaining an accurate T score for the corresponding thermographic image. Thus, the procedure for selecting these points and regions must be standardized and the protocol consistent.

At least two measurements are performed for all patients. The temperature difference between the nipple of each breast should be measured as well as the areolar temperature difference. Determining which temperature measurements to perform during analysis is a function of the appearance of particular qualitative patterns as described above and any functional abnormalities identified.

Thermography measures the temperature of the heat emitted from vessels of the skin. These vessels are controlled by the autonomic nervous system, specifically, the sympathetic nerves that innervate capillaries and blood vessels running through the layers of the skin and involved in thermoregulation. The exemplary autonomic challenge was placing the hands in cold water for 1 minute. The normal sympathetic response to the cold water autonomic challenge is vasoconstriction resulting in reduced emission of heat from the vessels graphically shown as an overall cooling in the images. A breast zone or thermovascular feature on a thermograph that shows increased heat emission following the autonomic challenge is considered to be abnormal. These functional analysis are an important part of the examination because some baseline thermographic images appear physiologically normal prior to the autonomic challenge, but have an abnormal response to the autonomic challenge. Only until comparisons between the Series 1 and Series 2 thermographic images are conducted are these abnormal responses apparent to the analyst.

Comparison of all points in a Series 1 thermograph with the same points in the corresponding Series 2 thermograph can highlight one or more points of interest that would warrant further quantitative analysis. Thus, the first step of image analysis can include a general point comparison to focus subsequent thermograph analysis to only those areas that responded abnormally to the autonomic challenge, i.e., points or regions that warmed in response to the cold water challenge. Alternatively, a hot spot or a global region in a Series 1 image can be selected and compared to a corresponding point or global region in the corresponding Series 2 image. If the hot spot remained unchanged or cooled slightly, it can be determined that there is no cause for concern or further analysis of that point. If the hot spot warmed, then it can be determined that this is an abnormal physiological response and that further quantitative analysis are warranted.

Using external photographs as a guide, a virtual line of symmetry can be taken from the patient's nose to their umbilicus. Quadrants or zones of each breast are also identified. Exemplary zones include upper outer, upper medial, lower outer, lower medial, and nipple/areolar zones of the breast. The point(s) selected for temperature measurement is based upon the presence of “hot spots” or unusual hypothermic spots in the thermograph, especially hot spots associated with a thermovascular feature identified during the qualitative assessment. For example, if a hot spot is located in the upper medial zone of the left breast, the hottest region within this hot spot is selected and its mirror point on the upper medial zone of the right breast is used for calculating the ΔT of that thermovascular feature or region of hyperthermia. In order to identify a “hot spot,” comparisons are made between the first and second series in the same thermal window to see if any vascular patterns or spots warm or cool. Similarly, comparisons from series 1 to series 2 are made with any asymmetrical pattern or spot to look for temperature change. Measuring mirror areas on the controlateral breasts is done if temperature differences appear to be 2° C. to 3° C. warmer than the opposite side.

Similar to the methods described above for the point temperature measurements, global regions can be selected to calculate average temperatures within a particular area of interest in the breast. The global regions selected can include an entire breast as well as breast zones such as the upper medial, medial, upper lateral, lateral, nipple and areolar zones of the breast. Global temperature measurement can be used, for example, to calculate the ΔT of a hot spot in the upper medial zone of the ipsilateral breast. In such an analysis, a point is selected at the hottest point of the hot spot within that zone. Then, a global region is drawn around the zone that includes the selected point of the hot spot. In this example, ΔT is the difference in temperature between the selected point and the mean temperature for the global region. If a temperature comparison with the contralateral breast is desired, mirror global regions on each side of the virtual line of symmetry are selected. The global regions must be as close to the identical in size as well as in mirror regions of the breasts. The mean temperatures for each global region are then used to calculate the ΔT. Standardization is primarily visual. Global measurements typically include the entire breast, but any smaller areas on the breast can be used. The size of the area is not so important as long as the area in question is the nearly the same on both sides.

Examples of four quantitative features that are primary risk factors for malignancy include regional hyperthermia that increases ≧0.5° C. following provocative testing (T score=40), nipple ΔT>2.5° C. (T score=50), areolar ΔT >2.5° C. (T score=50), and global hyperthermia ΔT>1.5° C. (score=50). If any of these features are identified, the patient should undergo follow-up thermographic examination and any appropriate additional diagnostic testing such as mammography, ultrasound, x-ray, MRI, sestamibi, ductal lavage, PET scan, blood tests, biopsy and the like.

A thermovascular pattern is a specific thermal pattern approximating the distribution of a blood vessel. These patterns of heat emissions appear on the thermographs, generally identified as vascular hyperthermia. The appearance of vascular hyperthermia in a breast calls for several different analysis to obtain ΔT values and generate a T score. The hottest point or “hot spot” of the identified thermovascular pattern is selected and the temperature difference between that hot spot to its mirror point on the contralateral breast is calculated. If ΔT between the hot spot and the mirror point on the contralateral breast is >2.0° C., the feature is characterized as T-1 and carries a score of 25.

The temperature difference of the hot spot to its surrounding area can also be calculated. To do this, the hottest point within the breast is selected and a global region around the hot spot is drawn. If ΔT between the hot spot and the mean temperature within the global region is ≧3.0° C., the feature is characterized as T-2 and carries a score of 20. It must be mentioned that the nipple/areolar zones should be excluded from these analysis as they carry a different weighted score and are part of a separate analysis to be described below.

The appearance of one or more well-defined areas of increased heat not necessarily associated with a thermovascular pattern is called regional hyperthermia. The regions can be large or small. The hottest point or “hot spot” of the identified hyperthermic region can be selected and the temperature difference between that hot spot to the mirror point on the contralateral breast can be calculated. A global region surrounding the hot spot can also be selected and the temperature difference between the mean temperature of the global region to the mean temperature of the mirror global region on the contralateral breast can be calculated. It is important to note that there can be more than one hyperthermic region and each region should be analyzed individually to calculate the ΔT. If ΔT between the hot spot and the mirror point on the contralateral breast or between the mean temperature of the mirror global regions is >2.5° C., the feature is characterized as T-3 and carries a score of 30.

The temperature difference of the hot spot to the mean temperature of its surrounding area on the ipsilateral breast can also be calculated. To do this, the hottest point within the hyperthermic region is selected and a global region around the hot spot is drawn. If ΔT between the hot spot and the mean temperature of the global region is >2.5° C., the feature is characterized as T-4 and carries a score of 25. Nipple/areolar zones should be excluded from these analysis as they carry a different weighted score and are part of a separate analysis.

An important step in determining risk of breast cancer is to calculate temperature differences between the nipple zones of the two breasts. Nipple temperature measurement is always performed during thermograph analysis. The center of the nipple zone of one breast is selected and the temperature difference with the mirror point on the contralateral breast is calculated. If ΔT between the hot spot and the mirror point on the contralateral breast is >1.0° C., the feature is characterized as T-5 and carries a score of 35. If ΔT between the hot spot and the mirror point on the contralateral breast is >2.5° C., the feature is characterized as T-6 and carries a score of 50. Such a score is primary risk factor associated with a very high correlation with breast carcinoma.

In addition to the differences in nipple temperature, areolar temperature measurement is also always performed during thermograph analysis. The areolar zone of one breast is selected and the temperature difference with the mirror point on the contralateral breast is calculated. If ΔT between the hot spot and the mirror point on the contralateral breast is >1.5° C., the feature is characterized as T-7 and carries a score of 35. If ΔT between the hot spot and the mirror point on the contralateral breast is >2.5° C., the feature is characterized as T-8 and carries a score of 50. Such a score is associated with a very high correlation with breast carcinoma. These scores are also a gold standard for raising concern for malignant breast disorders.

Global hyperthermia is a temperature pattern that includes the entire breast. For calculating global temperature differences, a global region is drawn over the entire breast showing the pattern of interest. A mirror global region of equal size is drawn on the contralateral breast. If ΔT between the mean temperature of the hyperthermic global region and the mean temperature of the mirror region on the contralateral breast is >1.5° C., the feature is characterized as T-9 and carries a score of 50. In addition to malignancy, other conditions that can cause global heat include abscess, papillomata, pericystic inflammation, fibroadenomata, mastitis, acute and chronic inflammation. This is a primary risk factor for malignancy.

Global hypothermia is also a temperature pattern that includes the entire breast and is characterized by unusually cold tissue. For calculating global temperature differences, a global region is drawn over the entire breast showing the pattern of interest. A mirror global region of equal size is drawn on the contralateral breast. If ΔT between the mean temperature of the hypothermic global region and the mean temperature of the mirror region on the contralateral breast is >2.0° C., the feature is characterized as T-10 and carries a score of 25. Conditions that commonly cause global hypothermia include cystic or fibrocystic breast changes.

The appearance of one or more well-defined areas of decreased heat not necessarily associated with a thermovascular pattern is called regional hypothermia. The coldest point or “cold spot” of the identified hypothermic region is selected and a global region surrounding the cold spot (ipsilateral breast) is selected. The temperature difference between the cold spot and the mean temperature of the global region surrounding the cold spot is calculated. It should be understood that there can be more than one hypothermic region and each region should be analyzed independently to calculate the ΔT. If ΔT between the cold spot temperature and the mean temperature of the surrounding global region is >2.0° C., the feature is characterized as T-11 and carries a score of 25. This finding seldom reflects malignancy. The analysis is reserved for patient experiencing local pain.

Comparisons between the Series 1 and Series 2 thermographic images are conducted to highlight any abnormal physiological responses. If ΔT between an area of regional hyperthermia in a Series 1 image and that same area in the corresponding Series 2 image is unchanged, the functional result is characterized as T-12 and carries a score of 20. However, if a Q-7 (regional hyperthermia) is used, then no score is assessed for a T-12 (as they represent essentially the same thing). If ΔT between an area of regional hyperthermia in a Series 1 image and that same area in the corresponding Series 2 image is between +0.1-0.4° C., the feature is characterized as T-13 and carries a score of 30. If ΔT between an area of regional hyperthermia in a Series 1 image and that same area in the corresponding Series 2 image >0.5° C., the feature is characterized as T-14 and carries a score of 40. This is a primary risk factor for malignancy.

The qualitative and quantitative image analysis described above as well as select portions of the patient medical history are used in generating a thermographic report 126. Report 126 can include standard patient data such as patient name, physician name, date of thermogram, a brief historical overview such as date of last diagnostic procedure(s), patient history and/or family history of cancer and the like. Report 126 can also include the Series 1 and Series 2 images used in the analysis and any information regarding the examination that were important to interpreting the images. Report 126 can also describe the analysis performed, as well as the Q scores and T scores obtained. Ultimately, the report provides a TH (thermal factor) value for each breast. In some implementations, information related to the analysis can be entered into processor 110, and processor 110 can be configured to forward the information to authority 103 to generate the report. Report 126 can then be sent back to, or accessed by processor 110, which can make the report available at an output, e.g., a display device, a printer, and the like.

The TH value provides the relative risk for the patient having a malignancy in that breast. The TH scoring system is based on original research conducted by M. Gauthrie, et al. (Thermal Assessment of Breast health, “Accurate and objective evaluation of breast thermograms: basic principles and new advances with special reference to an improved computer-assisted scoring system.” Publ MTP Press Ltd., Lancaster/Boston.) The thermographic findings were evaluated based on 26 standard thermological features and functional changes, during the course of the examination. Each of these was provided with numerical values or “scores”, which were developed based on the statistical frequency of occurrence and its association with breast pathology.

Q scores and T scores of all features present in each affected breast are calculated based upon the image analysis described above. Features such as asymmetrical, curvilinear, irregular, distorted, or anarchic thermovascular patterns as well as the ΔT for vascular hyperthermia, regional hyperthermia, nipple hyperthermia, areolar hyperthermia, global hyperthermia, global hypothermia, regional hypothermia, and the effect of autonomic challenge on the feature(s) are scored independently. Each feature identified carries with it a numerical score. Typically, one feature identified by itself is not considered the risk for breast malignancy. It is the cumulative score of all features that ultimately result in a TH value that indicates high risk. The numerical scores for each feature of one breast are added together for an overall score for that breast and the numerical scores of each feature of the other breast are added together for an overall score for that breast. These two cumulative scores are the TH values, one for the right breast and one for the left breast. The overall number of features identified and the weighted score for each of those features increases the patient's TH value for each breast and the risk for malignancy for each breast.

A cumulative Q/T score between 0-29 is characterized as TH-1. TH-1 is associated with relatively symmetrical and avascular patterns and considered statistically within normal limits regarding thermal emission. TH-2 indicates a cumulative Q/T score between 30-74 and is also within normal limits, but can be associated with benign breast disorders and occasionally early malignant changes. TH-3 indicates a cumulative Q/T score between 75-119 and is considered to be equivocal (inconclusive) or a patient at some risk for malignancy that should be followed closely. It is noted that early breast cancer and small breast carcinomas can be found with TH-3 scores, normally with scores above 80. TH-4 indicates a cumulative Q/T score between 120-149 and is considered to be abnormal and a patient at high risk for malignancy. TH-5 indicates a cumulative Q/T score greater than 150 and is considered to be abnormal and a patient at very high risk for malignancy.

Because different thermographic features can suggest a risk for different types of malignancies, the image analysis and patient report 126 serve as a guide the type of follow-up a patient receives. For example, nipple hyperthermia with a ΔT>2.5° C. has a very high correlation with ductal and lobular carcinoma. A patient diagnosed with nipple hyperthermia and scored T-6 and receiving a TH-3 value or higher would be referred for additional diagnostic workup such as mammography, ultrasound, etc. The TH value can provide information regarding the type of cancer by location, type of image presented and if patterns change rapidly or slowly. The report can help guide the type of treatment provided to the patient and allow for monitoring and evaluation of the patient to determine effectiveness of the selected treatment regimen as described in more detail below.

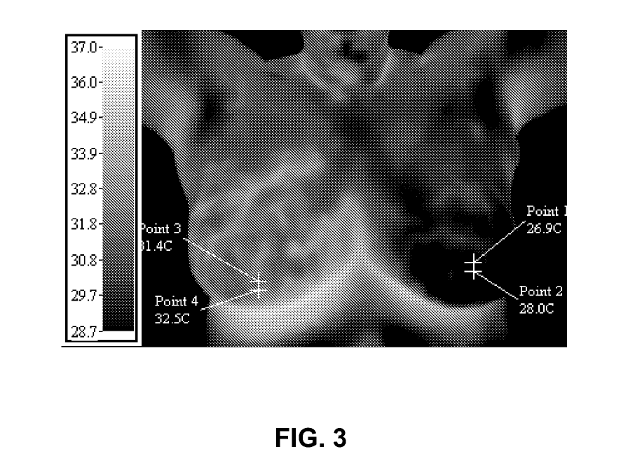

FIG. 3 shows an exemplary thermograph taken by digital infrared imaging, e.g., obtained via system 100. The patient's right breast shows increased chemical and blood vessel activity detected as heat in a thermograph as shown by regions of white or gray compared to the dark regions of normal breast tissue (left breast). Increased metabolic activity and increased vascular circulation (due to angiogenesis) in pre-cancerous tissue as well as any areas surrounding a developing cancer are often detectable using the system and methods described above, but \ cannot be detected by, e.g., mammography.

Breast examination and analysis was specifically described above; however, similar processes can be used to detect other disease processes, such as processes related to neurological disorders and vascular disorders to name but a couple.

As noted above, the images, scores, and report can be used to identify a possible treatment method when a risk of malignancy is detected. For example, since the TH score can be used to identify the type of cancer by location. Moreover, the process described above can be used in a clinical setting to determine whether certain types of treatments are effective in dealing with certain types of cancers at certain stages. Moreover, such clinical testing can reveal how a treatment progresses, its effects over time, how cancers normally respond to various treatments, etc. Armed with this clinical information, the TH score can be used for more than just determining a risk associated with the presence of various types of cancers. Now, the TH score can also be used to identify a treatment and to monitor the treatment progress.

For example, one identified reason for the occurrence of one type of breast cancer, namely intraductal carcinoma, is the deficiency of iodine in the body. Research indicates that areas in the breast where iodine is absorbed are the same areas where intraductal carcinoma starts in the absence of iodine. One reasons for a region of the breast to appear as a hot region during thermographic imaging is due the hot region being iodine deficient. In some implementations, a hot region of the breast can be identified by thermographic imaging techniques described previously. A Q score and a T Score can be assigned to the hot region and a TH score can be determined. Based on factors including the TH score of a hot region, an assessment can be made that the hot regions identified by thermographic imaging represent iodine-deficient areas. Subsequently, the patient can be given iodine-based medication to increase the quantity of iodine in the patient's body. Routine testing of the patient and regular thermal imaging of the hot region can be performed to monitor the patient's response to the iodine medication. As the iodine quantity in the patient increases, the thermal image of the hot region goes from hot to cold indicating a decrease in the iodine deficiency.

In some implementations, selenium-based medication can be added, as a supplement to the iodine-based medication, to improve, e.g., hasten, the decrease in temperature of the hot region.

Subsequent to thermographic imaging to identify hot regions in the breast, assessing that the hot region is due to a deficiency of iodine in the region, and providing the patient with iodine-medication, additional testing can be performed to determine the patient's responsiveness to the iodine-medication. In some implementations, the patient's urine can be collected at regular intervals for a period of 24 hours and the levels of iodine in the samples can be determined, e.g., using gas chromatography (GC) mass spectrometry.

In order to determine the ability of the patient's body to absorb iodine, a patient can be given a pre-determined quantity of iodine, e.g., 50 mg or 30 mg, and the patient's urine can be collected at regular intervals within a 24 hour period. The amount of iodine in the patient's urine can be used to determine the amount of iodine that was absorbed by the patient's body. Based on this determination, the patient's responsiveness to the iodine-medication can be determined.

Further, bromine/fluoride levels in the patient's body can be monitored over the course of the iodine-medication to assess the responsiveness of the patient's body to the iodine-medication. It has been observed that a body that is deficient in iodine absorbs other halogens, e.g., bromine, fluorine, from one or more sources, e.g., tooth paste, and the like. As the iodine levels in the body increase, the remaining halogens are displaced from the body. By monitoring a decrease in the levels of fluorine, bromine, and other halogens during the duration when the patient is on the iodine-medication, an increase in the level of iodine in the patient's body can be determined. Additional tests to assess iodine levels can include thyroid function tests.

Over the period that a patient is on the iodine-medication, thermographic imaging can be used to monitor any change to the hot regions that were identified prior to the patient being provided the iodine-medication. As the patient continues to take the iodine-medication, it can be expected that the temperature of the hot region, that is measured by the thermographic imaging techniques described previously, will decrease until the temperature becomes equal to or substantially close to normal body temperature. At this stage, it can be determined that the identified region is no longer iodine deficient. Despite the iodine-medication, if the temperature of the hot region does not decrease, it can be determined that iodine deficiency is not the cause for the temperature of the hot region, and alternative causes for the hot region can be identified

There are multiple lines of compelling evidence supporting the association between inflammation and cancer. Further, epidemiologic and clinical research indicates an increased risk of certain cancers in the setting of chronic inflammation. Many of the same processes involved in inflammation (e.g., leukocyte migration, dilatation of local vasculature with increased permeability and blood flow, angiogenesis) also contribute to tumor development. The ability of thermography to detect as heat the metabolic and physiologic changes involved in the initiation of a tumor provide the clinician and the patient an opportunity to intervene in the early stages of an evolving disease before a tumor develops. The patient can take appropriate preventative measures in order to avoid future development of a full-blown disease.

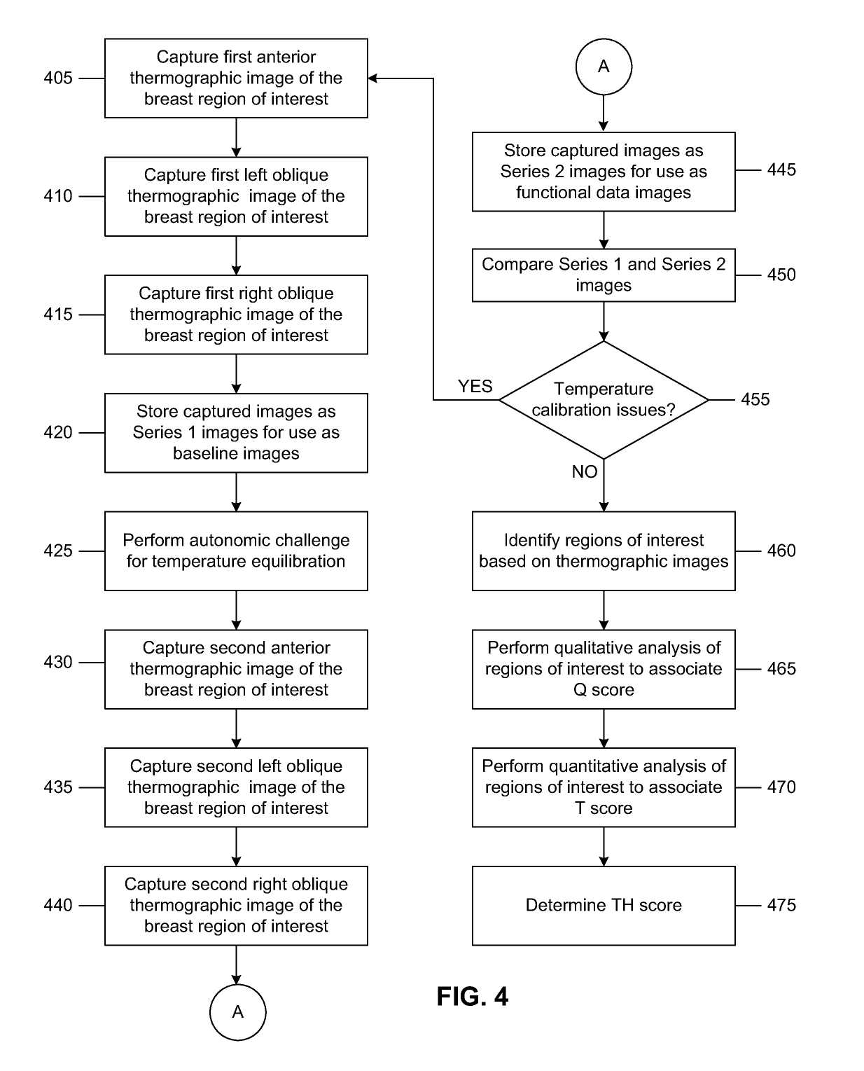

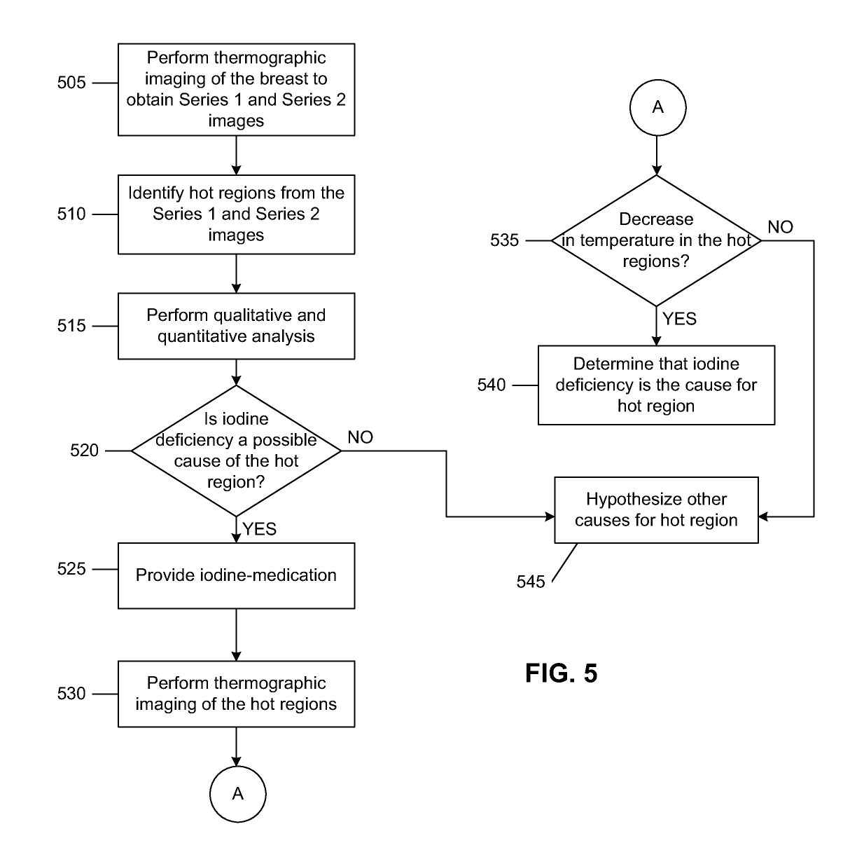

FIGS. 4 and 5 are flow charts illustrating how the process of risk detection, treatment identification, and treatment monitoring can work together using the systems and methods described herein and the results of various clinical analysis. FIGS. 4 and 5 are specifically directed to identification breast cancer risk and iodine treatment; however, it will be understood that similar system and methods can be used to identify risk, identify a possible treatment, and monitor the effects to the identified treatment with respect to other types of cancers, vascular disease, neurological disorders, etc.

FIG. 4 is an example of a process for the analysis of thermographic images of the breast region. The process can begin with the capture of a first anterior thermographic image of the breast region of interest at step 405. A first left oblique thermographic image of the breast region of interest can then be obtained at step 410, and a first right oblique thermographic image of the breast region of interest can be obtained at step 415. The captured images as Series 1 images can then be stored for use as baseline images at step 420.

The process can include performing an autonomic challenge for temperature equilibration, e.g., as described above, at step 425.

A second anterior thermographic image of the breast region of interest can then be obtained at step 430. A second left oblique thermographic image of the breast region of interest can then be obtained at step 235, and a second right oblique thermographic image of the breast region of interest can be obtained at step 240. The second set of images can then be stored as Series 2 images for use as functional data images at step 445.

In step 450, the Series 1 images and Series 2 images can be compared. After the initial comparison, a check can be performed to determine if there are temperature calibration issues based on the comparison at step 455. If there are temperature calibration issues, the process can re-start thermographic image capture at step 405, because the captured Series 1 and Series 2 images can no longer be used. If it is determined that there are no temperature calibration issues based on the comparison, then the process can continue with the identification of regions of interest based on the thermographic images. As describe above, a region of interest will often be a region where a temperature of the regions of interest is higher than the body temperature.

Qualitative analysis of regions of interest can then be performed to associate a Q score to the regions of interest at step 465. In some implementations, a grayscale version of the images can be analyzed to determine Q scores. Next, a quantitative analysis of regions of interest to associate a T score can be performed at step 470. In some implementations, color images can be analyzed to determine T scores. A TH score can then be determined based on the qualitative and quantitative analysis at step 475. In other words, as explained above, the TH score can be based on the determined Q score and the determined T score.

As assessment about iodine deficiency being a possible cause for the hot region can be made based on the TH score of the hot region. Such an assessment can be made to assist in the diagnosis of intraductal carcinoma. FIG. 5 is then a flow chart illustrating an example of a process for combining thermographic image analysis with iodine-based medication treatment. As a recap, the process can begin with thermographic imaging of the breast to obtain Series 1 and Series 2 images at step 505. Hot regions can then be identified from the Series 1 and Series 2 images at step 510. Qualitative analysis and quantitative analysis can then be performed at step 515 to associate Q scores and T scores, respectively, to the identified hot regions. An assessment can then be made to determine whether iodine deficiency is a possible cause of the hot region at step 520.

For example, such an assessment can be made to assist in the diagnosis of intraductal carcinoma. If it is determined that iodine deficiency can be one possible cause, then the process can include providing iodine-medication at step 525. The iodine medication can include over the counter iodine medication, prescription iodine medication, and supplements in addition to the iodine medication, e.g., selenium supplements. The process can include routinely performing thermographic imaging of the hot regions at step 530 to determine the responsiveness of the patient to the iodine medication.

Whether the temperature of the hot regions decreases can be determined at step 535 based on the images obtained in step 530. If it is determined that the temperature of the identified hot regions is decreasing, then the it can be concluded that iodine deficiency was the cause for the hot region at step 540. If thermographic analysis reveals that the temperature of the hot region does not decrease despite the patient being on the iodine medication, then it can be determined that iodine deficiency was not the cause of the hot spot and a new hypothesis for other causes for the hot region can be determined based on the TH score, etc. Also, new hypothesis can be offered or requested upon determining that iodine deficiency cannot be one cause of the hot region in step 520.

Again, as mentioned above, clinical analysis can be used to track a typical treatment process including how a cancerous, or pre-cancerous region reacts to various treatments. Thus, the analysis step 535 can comprise looking at more than just a decrease in temperature. Although, in the specific example of iodine treatment for intraductal carcinoma, a decrease in temperature has been identified as an indication of successful treatment at an early stage. Thus, the thermographic imaging of step 530 can be repeated over a period of time that has been identified as sufficient to show a decrease in temperature that should result from iodine treatment of intraductal carcinoma. If over this period of time no decrease in temperature, or an insufficient decrease in temperature, results, then it can be determined in step 540 that iodine deficiency is not the cause.

As noted above, while a patient is taking iodine medication, tests to determine the iodine levels in the body, e.g., GC mass spectrometry of urine samples, bromine/fluoride displacement tests, and the like, can be performed to determine if the level of iodine in the patient's body is increasing.

The thermographic imaging and thermographic analysis process can be performed by authority 103. A database 116 of previously collected images can be created. Q scores, T scores, and TH scores for each image can be stored in database 116. Authority 103 can be configured to compare thermographic images obtained from the patient and use the thermographic images stored in the database to determine Q scores, T scores, and TH scores for the captured images.

Another treatment that has been shown to be effective in reducing inflammatory, glycation, and oxidative stress factors within the pre-cancerous tissue, is the application of a detoxification through the skin using a dermal patch (described in more detail below). The dermal patch can be used to reduce inflammation. The patch can also be used to increase the antioxidant reserves, tissue repair capabilities, and acid-base balance in the breast tissue of a woman identified via thermography as having pre-cancerous tissue. Reducing risk factors that promote an evolving disease process or pre-cancerous condition into an active cancer can prevent or delay the development of the disease.

Detoxification patches act like a poultice to remove toxins from the body through the skin. A poultice can be made of a porous material. A solvent of the poultice equilibrates with target solute in a body and by passive diffusion solute enters the poultice through the skin thereby having “detoxifying” effects. After an adequate time passes for this process to occur, the poultice is removed and with it the dissolved solutes or toxins.

The patch can be, for example, a pouch or other sealed enclosure or bag formed of a permeable fabric such as gauze, muslin, linen, or white cotton sheeting. The size and shape of the patch can vary. The ingredients that permeate the patch can provide treatment that reduces risk factors associated with the progression of the evolving disease process. For example, the ingredients in the patch can provide anti-inflammatory and anti-oxidant properties. Similarly, ingredients in the patch can receive or extract toxins from the body such as heavy metals, free radicals and chemicals from the body. Thus, the mesh-like or porous patch can act in a uni-directional or bi-directional manner.

The ingredients of the patch can include, but are not limited to one or a combination of: a mineral, a silicon-based mineral, a far-infrared emitting element, clay, tourmaline, citrine, wood vinegar, bamboo vinegar, vitamin C, dokudami, loquat leaf, amygdalin, vitamin B17, laetrile, chitosan, chitin, turmeric, curcumen, milk thistle, pau d'arco. The patch may include powdered tourmaline crystal, bamboo vinegar, vitamin C, dokudami, loquat leaf, and chitosan. An exemplary mixing ratio of the above ingredients can be as follows (% weight): tourmaline crystal 30%, bamboo vinegar 21%, wood vinegar 20%, chitosan 1.5%, loquat leaf 1.5%, dokudami 7%, Vitamin C 1.5%, vegetable fiber 7.5% and dextrin 10%. The ingredients of the patch can vary according to the application of the obtained composition. For example, additional active ingredients such as curcumen, milk thistle and/or pau d′arco can be added to the above ratio of ingredients.

Silicon-based minerals such as granite, perlite, pitchstone, and tourmaline can be used as main components. These minerals radiate electromagnetic waves (feeble energy) and release anions. The mineral in the patch can be tourmaline. Tourmaline is both pyroelectric and piezoelectric. If a specimen is put under pressure, or a temperature change, it will generate an electrical charge. Tourmaline is best known as one of the only minerals to emit far infrared heat and negative ions. Pyroelectricity of tourmaline results in adsorbing properties such as fixing heavy metal ions and adsorbing malodorous composition particles. Tourmaline can be milled into a powder using No. 40 mesh. For example, the particle size of the powdered tourmaline crystal using 40 mesh can be the size of 420 μm. The mineral in the patch can be a multi-elemental mineral, such as a mixture of tourmaline and citrine. The mineral powders can be used without further processing. Alternatively, the powders can also be used after they are mixed with water, whether heated or pressurized, so that the clear liquid part of the water dries into a powder by vacuum-freeze drying or by spray drying methods.

Bamboo vinegar is a material analogous to pyroligneous acid. It represents the upper part of the liquid obtained by cooling the gas generated in a process of dry distillation of bamboo or in a process of producing bamboo charcoal, as in pyroligneous acid (i.e., wood vinegar). It contains acetic acid and methyl alcohol. The substance has sterilization, deodorization and humidity conditioning effects due to its excellent adsorbability.

Other ingredients can be selected based upon their anti-inflammatory, anti-oxidant and anti-cancer properties. For example, Vitamin C (ascorbic acid) can be obtained from citrus extracts such as grapefruit extract or orange extract. Vitamin C and citrus extracts provide anti-oxidant properties and anti-mutagen properties. The scent of the citrus extracts provides a pleasant aromatherapy. Dokudami (houttuynia herb) is a plant known to have strong adsorption properties. Loquat leaf (Eriobotrya japonica Lindley) contains malic acid, tartaric acid, citric acid tannate, carotene, vitamins A, B and C. Its leaves are mainly used for their anti-inflammatory effect. They also contain amygdalin (vitamin B17), which is also known as the anti-cancer vitamin. The rhizome (root) of turmeric (Curcuma longa Linn or curcumin) has anti-inflammatory, antioxidant, and anti-cancer properties. Milk thistle has been shown to provide anti-inflammatory properties and is also known for its beneficial effects on general breast health in females. Pau d'arco has been used in South America as a cancer treatment for several decades. It is also known as taheebo, ipe roxo and cancer tea. Chitosan can be obtained from chitinous substance included in carapaces of conchostracan such as prawn and crab. Chitosan products have been used by water companies to trap toxins, grease, heavy metals and oils. Chitosan is also used in the medical profession to promote wound healing of burns and skin inflammation.

As discussed above, thermograms detect abnormal temperature patterns indicative of the presence of inflammation and an evolving disease process. Studies have shown a relationship between microvessel density and thermographic hot areas surrounding breast tumors. Information on this process can be found in, for example, Yahara et al. Surg Today 33(4):243-8 (2003), which is incorporated herein by reference as part of this specification. As shown in FIG. 3, the chemical and blood vessel activity in pre-cancerous tissue (right breast) and the surrounding areas of the developing breast cancer is higher than in the normal breast tissue (left breast) and is detected as heat or hot spots in a thermograph. The physiologic information provided by thermograms allow for the detection of pre-cancerous areas of the breast tissue. In turn, the opportunity is provided to take appropriate preventative steps in order to avoid the development of full-blown disease by reducing certain risk factors associated with the progression of the evolving disease process.

It has been suggested that one of the first biochemical signals of change in breast cells may be expressed as an inflammatory response. Further, a theoretical model of the inflammatory process has been suggested and predictive linkages shown among stimuli in the breast microenvironment and the development of breast cancer. Information on this process can be found in, for example, Lithgow et al., Biol Res Nurs. 7(2):118-29 (2005), which is incorporated herein by reference as part of this specification. We provide here a method of anti-inflammatory therapy by a dermal detoxification patch to prevent or delay progression of pre-cancerous tissue identified by thermographic imaging into diseased tissue.

Thus, in another exemplary treatment protocol, one or more detoxification patches are positioned on the skin covering regions of the breast identified by thermography as expressing or having abnormal temperature patterns or temperature patterns indicative of an evolving disease process. The positioning of the patch is assisted by or is performed according to the thermographic map of pre-cancerous regions or hot spots provided by the infrared imaging system. The patches are placed into direct contact with the targeted portion of the skin where care or treatment thereof is desired. For example, the regions of skin directly overlying tissue expressing the abnormal temperature pattern can be located according to the thermographic image and the treatment device positioned on that region or those regions of skin. The patch(es) can be fixed in place by an adhesive bandage.

Treatment protocols can vary depending on the size and severity of pre-diseased regions identified. It should be noted that use of the patches is also not limited to only the breast, but can be applied to any body part. For example, additional patches can be positioned according to reflexology points on the patient's foot. Treatment protocols can vary also with respect to number of days and the length of time per day the patch is positioned on the patient's skin. Patches can be used daily between, for example, around 5 to around 15 hours per day. The course of treatment can be, for example, between around 5 days up to around 90 days. One exemplary treatment protocol includes treatments between around 7 to around 10 hours nightly while the patient is sleeping, for a minimum of 5 days. Patches are removed each morning and can be saved for further analysis (described in more detail below). The detoxification patch can be part of a kit. The kit can include at least one patch enclosed in a protective wrapper, at least one adhesive sheet covered by backing paper for adhering the patch to the patient's skin. Each kit can contain the appropriate number of patches and adhesive sheets needed for a course of therapy (i.e. number of days of therapy would be equal to the number of patches in the kit).

Following the treatment protocol, patients can undergo follow-up thermographic imaging. The images obtained can provide evidence of reduction in inflammation or “hot spots” due to treatment with the patches. Further thermographic images can be obtained to monitor lasting efficacy of treatments and, if applicable, additional courses of treatments. In addition, correlative lab values known to identify inflammatory processes can be performed on patients prior to and following treatment with the detoxification patches.

The used detoxification patches can be analyzed to assess efficacy of treatment. For example, presence of factors related to inflammation found in the used detoxification patches can be analyzed by methods known in the art. Similarly, removal of other factors, such as toxins and heavy metals, can be analyzed by known methods. For example, levels of benzene, nickel, isopropyl alcohol, thallium, methyl alcohol, thulium, aluminum, arsenic, cadmium, asbestos, copper, azo dyes, lead, PCBs, and mercury collected in the patch after use can be analyzed by methods known in the art. Similarly, hair samples taken prior to and after treatment can be analyzed to show reduction in selected toxins and heavy metals according to methods known in the art. Dark field microscopy can also be used to identify other items extracted from the tissues. The analysis of used detoxification patches can help to determine what the next step of treatment can be or what other preventative measures can be taken. The presence of factors identified on the used patches could also indicate the early stages of other types of disease processes not yet identified.

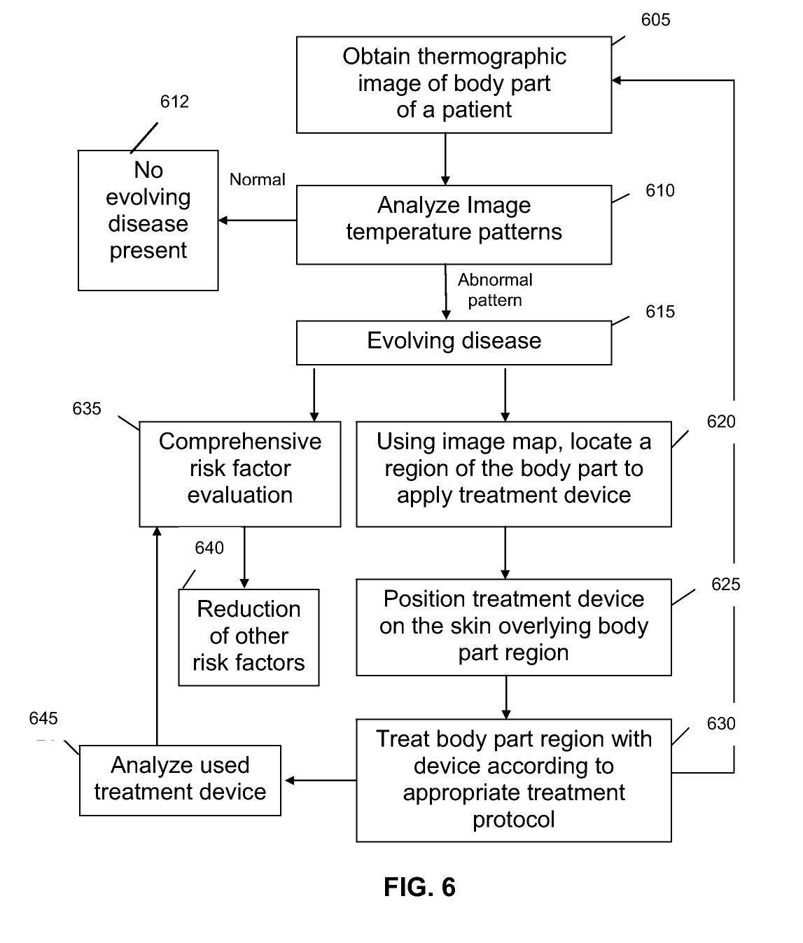

FIG. 6 is a flowchart illustrating one exemplary method of treatment. In this method of treatment a thermographic image of a body part of a patient is obtained at step 605, and temperature patterns of the thermographic image(s) are analyzed in step 610 as described above. If abnormal temperature patterns are found it can be indicative of an evolving disease process as determined in step 615. Once the abnormal thermogram is identified, patients can undergo a two-prong approach of disease prevention.

One aspect of the two-prong approach can include a detoxification patch treatment protocol. The thermographic image showing abnormal regions of heat emission can be used like a map to locate a region of the body part on which to apply the treatment device or detoxification patch in step 620. Once a region of the body part is identified, the treatment device is positioned on the skin overlying the target region in step 625. The device can be affixed to the skin by methods known in the art, such as an adhesive strip or bandage or the like. The body part region is then treated with the device according to a treatment protocol in step 630 such as those protocols described above. Following treatment with the device, the patient can undergo follow-up thermographic imaging to assess efficacy of treatment in step 605.

Another aspect of the two-prong approach can include placing patients on a comprehensive evaluation of risk factors in step 635. The evaluation can include assessing hormone levels, nutrient and dietary intake levels, intestinal tract health, metabolic health, glycation, inflammation, oxidative stress, antioxidant reserves, tissue repair deficits, acid-base balance, stress and psychological factors, heavy metal toxicities and exposure to pollutants. The used treatment patches also can be analyzed in step 645 to help identify and evaluate risk factors of the evolving disease process. Based on the findings of the analysis of used patches as well as the results of the comprehensive risk factor evaluation, supplemental treatment(s) can be performed and/or lifestyle changes made to reduce the identified risk factors of the evolving disease process in step 640.

It should be noted that use of the detoxification patches is not limited to treatment of inflammation and pre-cancerous tissue. Information on the 300 chemical pollutants in breastmilk can be found in, for example, http://www.foeeurope.org/publications/2006/toxic_inheritance.pdf, June 2006, which is incorporated herein by reference as part of this specification. Thus, these detoxification patches can be used to remove long-lived toxins, lipophilic chemicals and heavy metals from breastmilk of lactating women. Similarly, the detoxification patches can be used in women who are pregnant or plan to become pregnant to prevent transmission of these toxins through the placenta to the fetus.

Returning to FIG. 1, authority 103 can be configured to automate the above analysis, scoring, and report generation. In certain embodiments, processor 110 can be configured to provide dynamic information to authority 103 as opposed to static information as in conventional systems. This, for example, can allow authority 103 to have access to 7600 unique temperature points in each image as well as a resolution of 1/10 of a degree. With this much information in each image, authority 103 can be programmed, e.g., via modules 118 to perform automatic qualitative analysis and score generation. For example, authority 103 can be programmed to automatically make certain qualitative measurements and comparisons, such a left whole breast temperature, right whole breast temperature, and the delta there between.

The qualitative measurements can then be used by authority 103 to generate the Q scores 120 described above.

Similarly, authority 103 can be configured to automatically make quantitative measurements as well. The quantitative measurements often include, or are based on some sort of pattern recognition. Thus, authority 103 can include digital signal processing, neural network, graph theory based modules 118, or some combination thereof, in order to allow authority 103 to recognize certain patterns and generate the T scores 122 describe above.

In certain embodiments, a technician can also review the images in order to help with the quantitative analysis. In order to aid the technician, authority 103 can be configured to display the images in color so that the temperature patterns can be detected. Further, the display can include a control mechanism that allows the technician to select the temperature window and thereby control the range of temperature data being displayed for each image. For example, the default range maybe 10 degrees. Thus, the technician can select 10 degree ranges of temperatures to be displayed. In certain embodiments, for example, the display can include a slider control that allows the technician to select, or change the range of temperatures by sliding the control mechanism.

This allows patterns to be detected from a smaller range of data. If data for the full temperature range is included, in may prevent automated, or even manual detection of patterns of interest. The ability to select a smaller range of temperature data can improve the ability to detect patterns of interest.

The left side of FIG. 3 illustrates a range of temperatures being provided in the associated image. Thus, in certain embodiments, this range of temperatures can be dynamic and allow the selection of different ranges.

With the Q scores 120 and T scores 122, authority 103 can be programmed to automatically generate the TH scores 124 and to generate a report 126. As noted above, the reports can be stored in database 116 and made available via processor 110, e.g., via a thin client running on processor 110. Alternatively, the report can be sent back to processor 110 via network 112.

It should be noted that images saved in database 110 can be used in conjunction with new images to determine whether a proscribed treatment is working, or effective. For example, when images are taken after a treatment, e.g., in step 530, those images can be compared to previous images, e.g., images taken in step 505, as well as to each other. Changes in temperature or patterns between images taken prior to treatment, or previously during treatment, can then be correlated with a score, or scores that can be used to determine whether the treatment is effective or not. Again, this type of analysis and scoring can be performed automatically by authority 103.

While certain embodiments have been described above, it will be understood that the embodiments described are by way of example only. Accordingly, the systems and methods described herein should not be limited based on the described embodiments. Rather, the systems and methods described herein should only be limited in light of the claims that follow when taken in conjunction with the above description and accompanying drawings.

What is claimed is:

1. A system for detecting the risk of breast cancer, comprising;
a thermographic imaging device configured to acquire thermographic images of a female torso;
an analysis authority communicatively coupled with the thermographic imaging device, the analysis authority including:
an input port configured to receive thermographic images from the thermographic imaging device,
a database configured to store the thermographic images,
a qualitative analysis module configured to automatically perform a qualitative analysis of the thermographic images and generate a qualitative score based on the qualitative analysis,
a quantitative analysis module configured to automatically perform a quantitative analysis of the thermographic images and generate a quantitative score based on the quantitative analysis, and
a scoring module configured to correlate the qualitative and quantitative scores with a score indicating a risk of breast cancer.

2. The system of claim 1, wherein the thermographic imaging device is configured to acquire a first series of images and a second series of images, and wherein the qualitative analysis is performed based on comparisons between the two image series.

3. The system of claim 1, wherein the thermographic imaging device is configured to acquire a first series of images and a second series of images, and further comprising a comparison module configured to compare the two image series and to identify the areas of interest based on the comparison, and wherein the qualitative and quantitative analysis modules are configured to perform the qualitative and quantitative analysis on the areas of interest.

4. The system of claim 2, wherein the second series of images are acquired after an autonomic challenge.

5. The system of claim 3, wherein the comparison module is further configured to determine whether a temperature calibration issue exists based on a comparison between the two series of images.

6. The system of claim 1, wherein the thermographic images include a anterior image of a breast region of interest, a left oblique image of the breast area of interest; and a right oblique image of the breast area of interest.

7. The system of claim 1, wherein the analysis authority is further configured to receive and store photographs of the female torso, and to use the photographs to identify areas of interest in the thermographic images.

8. The system of claim 1, further comprising a report module configured to automatically generate a report based on the out put of the qualitative analysis module, quantitative analysis module, and scoring module.

9. The system of claim 1, further comprising a cancer identification module configured to identify risk of a certain type of cancer based on the output of the qualitative analysis module, quantitative analysis module, and scoring module.

10. The system of claim 1, further comprising a treatment identification module configured to correlate the output of the scoring module with a specific treatment.

11. A method for detecting the risk of breast cancer, in a system comprising a thermographic imaging device configured to acquire thermographic images of a female torso and an analysis authority communicatively coupled with the thermographic imaging device, the method comprising:
receiving thermographic images from the thermographic imaging device;
storing the thermographic images in a database;
automatically performing a qualitative analysis of the thermographic images;
generating a qualitative score based on the qualitative analysis;
automatically performing a quantitative analysis of the thermographic images;
generating a quantitative score based on the quantitative analysis; and
correlating the qualitative and quantitative scores with a score indicating a risk of breast cancer.

12. The method of claim 11, further comprising acquiring a first series of images and a second series of images and comparing the two sets of images, and wherein the qualitative analysis is based on the comparison between the two image series.

13. The method of claim 11, further comprising identifying areas of interest in the thermographic images, and wherein the qualitative and quantitative analyses are performed on the areas of interest.

14. The method of claim 12, further comprising performing an autonomic challenge, and wherein the second series of images are acquired after an autonomic challenge.

15. The method of claim 12, further comprising determining whether a temperature calibration issue exists based on a comparison between the two series of images.

16. The method of claim 11, wherein the thermographic images include a anterior image of a breast region of interest, a left oblique image of the breast area of interest; and a right oblique image of the breast area of interest.

17. The method of claim 11, further comprising receiving and storing photographs of the female torso, and using the photographs to identify areas of interest in the thermographic images.

18. The method of claim 11, further comprising automatically generating a report based on the qualitative analysis, quantitative analysis, and score indicating a risk of breast cancer.

19. The method of claim 11, further comprising identifying risk of a certain type of cancer based on the qualitative analysis, quantitative analysis, and score indicating a risk of breast cancer.

20. The method of claim 11, further comprising correlating the score indicating a risk of breast cancer with a specific treatment.
